(19) United States
(12) Patent Application Publication
(10) Pub. No.: US 2022/0016163 A1
(43) Pub. Date: Jan. 20, 2022

(54) DENDRITIC HUMAN UNIVERSAL NON-ANTIGEN EDUCAP VACCINE

(71) Applicant: William Richard Deagle, Vista, CA (US)

(72) Inventor: William Richard Deagle, Vista, CA (US)

(21) Appl. No.: 16/948,930

(22) Filed: Oct. 6, 2020

Related U.S. Application Data
(60) Provisional application No. 62/705,877, filed on Jul. 20, 2020.

(51) Int. Cl. A61K 35/15; C12N 5/0784

(52) U.S. Cl. CPC A61K 35/15; C12N 5/0639

(57) ABSTRACT

A non-antigenic microRNA exosomal vaccination that includes dendritic-derived exosomes resulting from the incubation of dendritic cells with Spike Protein of a COVID-19 virus so that the dendritic cells process the Spike Protein of the COVID-19 virus to enable education of naïve T-cells. The exosomes so-derived are then harvested and encapsulated for delayed release oral capsule delivery to selectively reach intestinal antigen-presenting cells in the terminal ileum for stimulating an acquired immune response to the Spike Protein of the COVID-19 virus. The intestinal antigen-presenting cells incorporate the dendritic-derived exosomes, and thereby learn to recognize the Spike Protein as a threat, even though no dangerous antigens, RNA, or DNA modifications of the antigen or Spike Protein of the COVID-19 virus were introduced by the non-antigenic microRNA exosomal vaccine, thereby avoiding creation of new pandemic viral emergencies, cytokine storms, mitochondrial aerobic failure, serious illnesses, and death via mutated strains of the COVID-19 virus.

### CROSS-REFERENCE TO RELATED APPLICATIONS

The present application claims priority from U.S. Provisional Patent Application 62/705,877 filed Jul. 20, 2020, the contents of which are herein incorporated by reference.

### FIELD OF THE INVENTION

The present invention relates generally to immunotherapy, and more particularly to an exosomal biologic and related methods for conferring T-cell and B-cell immunity.

### BACKGROUND OF THE INVENTION

Known prophylactic vaccines, when successful, activate acquired immunity to a particular infectious disease through preparation of a biological agent that resembles a disease-causing microbe. The biological agent may be a weakened or killed form of the microbe, its toxins, or one of its surface proteins or antigens. The aim of the prepared biological agent is to stimulate the body's immune system to recognize the biological agent, and thereby recognize the underlying disease-causing microbe as a threat, thereby preventing or ameliorating the effects of a future infection. Vaccines can also be therapeutic so as to fight a disease that has already occurred.

Current messenger RNA (mRNA) vaccines, such as the family of vaccines that are used to fight severe acute respiratory syndrome coronavirus (SARS-CoV-2), and which are the current focus of COVID-19 vaccine research, create binding antibodies that do not activate T-cell viral killing capacity, and that do not block viral ACE2 receptor entry. Furthermore, current mRNA vaccines can create excessive free radicals and cytokines on exposure to the target virus, placing the host in great danger. That is why studies have shown that past mRNA vaccines for SARS1, MERS, and Gardasil have all proved to be useless and dangerous, as they produced toxic cytokine reactions, autoimmunity, neurological syndromes, and in some cases, cardiorespiratory arrest and death. Moreover, mRNA vaccines for use against COVID-19 are likely to increase autoimmunity and future cancer risks with genetic host modification. As a result, the multitude of developing vaccines may cause new pandemic viral emergencies, cytokine storms, mitochondria aerobic failure, serious illnesses, or death. They will offer no protection against future viral cell entry, nor will they stop mutant COVID-19 strains from causing further illness and death.

### SUMMARY OF THE INVENTION

According to the invention, incubation of dendritic universal human cell culture (DUHCC) with Spike Proteins of COVID-19 can be used to create and extract resulting immune educated exosomes therefrom. Then, these immune educated exosomes, which are permanent transferable immune memory molecules, are delayed-release encapsulated and orally delivered to in turn educate/activate T-cells and B-cells against the antigens of COVID-19 used to create the initial immune educated exosomes. The thereby-educated cells in turn produce exosomes that further transfer the T-cell immune activation education to other cells.

In immunology, an antigen is a molecule or molecular structure of a disease-causing microorganism that can be bound to by an antigen-specific antibody or B-cell antigen receptor or T-cell receptor, which are key cellular components of adaptive immunity. The presence of antigens in the body normally triggers an immune response, which is the aim of the known prophylactic vaccines.

Antigens are molecules that bind to specific receptors, and thus can be “targeted” by antibodies created by the immune system. An antibody, in other words, is a feedback response of the immune system. The antibody is specifically produced by the immune system to match an antigen after cells in the immune system come into contact with the antigen. This contact allows a precise identification or matching of the antigen, and the initiation of a tailored “acquired” immune system response to the antigen. The antibody is said to “match” the antigen in the sense that it can bind to the antigen via receptors on a portion of the antibody. However, an antigen cannot necessarily induce an immune response in the body by itself; an immune response activation requires presentation of antigen-presenting cells.

Dendritic cells are the most efficient cells at presenting antigens, enabling activation of naïve T-cells, and initiating the adaptive immune response to the antigen and the associated pathogen. In short, dendritic cells are antigen-presenting cells of the mammalian immune system. Their main function is to process antigen material and present it on the cell surface to the T-cell of the immune system. Thus, dendritic cells act as messengers within the adaptive immune system.

Dendritic cells are present in those tissues that are in contact with the external environment, such as the skin, and the inner linings of the nose, lungs, intestines, and stomach. They can also be found in an immature state in the blood. Once activated, they migrate to the lymph nodes where they interact with the T-cells and the B-cells to initiate and shape the adaptive immune response.

Most pathogens colonize and invade the host at mucosal surfaces, such as the lungs and the intestines. To combat intestinal pathogens, induction of local adaptive immune responses is required, which is mainly achieved through oral vaccination. However, most known vaccines are ineffective when given orally, owing to the hostile environment in the gastrointestinal tract. Further, the uptake of such known vaccines by intestinal immune cells is typically rather poor.

Extracellular vesicles (EV) are small phospholipid bilayer vesicles, released byall prokaryotic and eukaryotic cells, which can contain different types of RNA, proteins, mitochondrial DNA, and both single-stranded DNA and double-stranded DNA, spanning all chromosomes. Exosomes are one type of EV. Exosomes are defined as being tiny membrane-bound particles ranging in size from 30 nm to 150 nm.

Originally dismissed as merely a cellular mechanism for waste disposal, exosomes are now viewed as important players in intercellular communication, especially because of the protein and micro-RNA cargo that is typically packed into these particles.

The present invention employs dendritic universal human cell culture that is incubated with antigens of the Spike Proteins of COVID-19 to educate the dendritic cells so as to create exosomes of the presentation-enabled dendritic cells. These dendritic-derived exosomes contain permanent transferable microRNA memory molecules that can be delivered orally to further educate and thereby arm the human immune system against COVID-19. This education modulates and terminates autoimmunity caused by COVID-19, and alerts the immune system to seek and destroy COVID-19 viruses.

The present invention is safe, because no viral pathogenic antigens (e.g., Spike Proteins), RNA modifications, or DNA modifications to generate antigens in the host to return the host to a balanced alert appropriate immune state, without immunological dysfunction. It takes only five days of incubation, on average, to create trained dendritic immune memory cells (DIMC). This generates the exosomes containing microRNA that are freeze-dried and then DR (delayed release) encapsulated in the EduCap™, the microRNA containing the immune information that is used by the body to create natural immunity.

Following the administration of the orally delivered vaccine of the invention, and the subsequent passage of the microRNA-containing exosomes through the M cell compartment of specialized cells in the terminal ileum. Thereby, the exosomes are delivered to dendritic cells in the liver, spleen, and bone marrow. The individual's immune system uses the microRNA of the exosomes to store the immune memory and protect a T-cell and B-cell response leading also to specific immunoglobulin A (IgA) and IgG and later IgM production and secretion. The unique effect of this oral vaccine (EduCaps™) is that it blocks COVID-19 and all variants by targeting the ACE Receptor Key that enables COVID to enter the human cell. The EduCap™ targets the Receptor Binding Domain portion of the Sigma One portion of the Spike Protein, which is common to ALL variants of COVID-19. Any variation of COVID-19 having a change to the RBD will NOT be infective to humans, and is thereby harmless.

Thus, the invention provides dendritic-induced exosomes, which contain human, non-antigenic microRNA memory molecules, wherein the dendritic-induced exosomes are encapsulated, and then orally delivered so as to selectively target intestinal exosome-receiving cells in the terminal ileum, which then go to the liver sinusoids, spleen, and bone marrow. This oral delivery system of vaccination is referred to herein as “EduCaps™”, i.e., vaccination by capsule-education of an immune system.

All current known injectable vaccines produce cytokine storms and toxic free radicals, insert mRNA, as well as introduce toxic adjuvant chemicals and heavy metals. By contrast, the oral capsule delivery of dendritic-induced exosomes of the invention avoids all these toxic effects, while upgrading immunological surveillance and protection sub-programs, non-toxically.

The present invention further embodies an oral immune education system against COVID-19 and all variants thereof, with no toxic immune imbalances, no gene insertions, no toxic heavy metals or adjuvants. Nevertheless, the invention provides robust viral receptor-blocking of the Sigma One entry point of the Spike Protein into ACE2 receptors of human cells, thereby blocking infection, which results in the additional benefit of effectively preventing spontaneous or vaccine-induced future mutant versions of COVID19.

A general aspect of the invention is a method of vaccinating a person against COVID-19 and all variants thereof. The method includes: incubating a dendritic cell culture of a receptor binding domain of a Sigma One portion of a spike protein of a COVID-19 virus, or any variant of a COVID-19 virus; separating a plurality of microRNA exosomes from the dendritic cell culture; and administering the plurality of microRNA exosomes to the person, the plurality of microRNA exosomes conferring to the person T-cell and B-cell immunity against COVID-19 and all variants of COVID-19.

In some embodiments, the plurality of microRNA exosomes is encapsulated.

In some embodiments, administering the plurality of microRNA exosomes to the person includes encapsulating the plurality of microRNA exosomes in a delayed release capsule so as to target intestinal antigen-presenting cells in the terminal ileum.

Another general aspect of the invention is a composition for vaccinating against COVID-19. The composition includes: a plurality of dendritic-derived exosomes isolated from a plurality of dendritic cells incubated with Spike Proteins of COVID-19 viruses.

Known mRNA vaccines include protein-generating mRNA for generating the entire Spike Protein. By contrast, the invention uses microRNA (miRNA) for immune education to recognize the Receptor Binding Domain (RBD) portion of the Sigma One Portion of the COVID-19 Spike Protein, and then to terminate COVID-19 viruses, and variants thereof.

### BRIEF DESCRIPTION OF THE DRAWINGS

The invention will be more fully understood from the following detailed description, in conjunction with the following figures, wherein:

FIG. 1 is a flowchart of an exemplary embodiment of the present invention;

FIG. 2 is a flowchart of an exemplary embodiment of the present invention;

FIG. 3 is a diagrammatic view of pandemic-induced chaos which the present invention aims to avert;

FIG. 4 is a schematic view of the dendritic culture Sigma One receptor ACE2 protein;

FIG. 5A is a schematic view of an exemplary embodiment of the present Invention;

FIG. 5B is a schematic view of an exemplary embodiment of the present invention;

FIG. 6 is a schematic view of the trained Sigma One exosomes released in culture;

FIG. 7 is a schematic view of the Sigma One liposomes packages in micelles;

FIG. 8 is a schematic view of the exosomes neurological memory solid state immunity T-cells and B-cells;

FIG. 9 is a schematic view of an exemplary embodiment of the present invention;

FIG. 10 is a schematic view of an exemplary embodiment of the present invention;

FIG. 11 is a schematic view of a Delayed Release oral capsule;

FIG. 12 is a schematic view of the SAFE GI transfer EduCaps™ to acceptor T-cells and B-cells;

FIG. 13 is a schematic view of the exosome micro-vesicle's solid state immunity molecules;

FIG. 14 is a diagrammatic view of the systems dynamic memory immunity;

FIG. 15 is a diagrammatic view of the vaccine trail steps;

FIG. 16 is a diagrammatic view of an exemplary embodiment of the present invention;

FIG. 17 is a diagrammatic view of the return to an open world;

FIG. 18 is a diagrammatic view of the U.S. public private partnership;

FIG. 19 is a diagrammatic view of the post pandemic open safe world; and

FIG. 20 is a diagrammatic view of an exemplary embodiment of the present invention.

### DETAILED DESCRIPTION

Broadly, the present invention provides a non-antigenic vaccination composition, method, and system that embodies dendritic-derived exosomes resulting from the incubation of dendritic cells with the Spike Protein of COVID-19, the Spike Protein including an RBD portion that is common to ALL variants of COVID-19 in such a way that the dendritic cells process the Spike Protein of COVID-19 for presentation so as to enable immune education of naïve T-cells and B-cells. The exosomes derived from those incubated dendritic cells are harvested and encapsulated for oral delivery so as to be released within the terminal ileum having exosome receptor cells. The exosome receptor cells transfer the exosomes to the splanchnic circulation to the liver sinusoids, spleen, and bone marrow, thereby conferring an acquired immune response to the COVID-19 virus, and all variants thereof. Acquiring an immune response to the COVID-19 virus (or any variant), in the manner according to the invention, does not expose the person to the COVID-19 virus, or any dangerous antigens thereof.

The EduCaps™ of the invention do not cause the person's cells to create Spike Proteins for immune education. Instead, EduCaps stimulates both cellular T-cell viral killing, and humoral B-cell viral identification immunity. By contrast, all known mRNA and DNA vaccines only generate partial B-cell viral identification (non-killing) immunity. For example, the partial B-cell viral immunity does not stop other sub-strains, such as the Delta variant and the Mu variant. Further, EduCap™ vaccination against COVID-19 does not create new viral strains that can evade EduCap™ immune identification and elimination. By contrast, known mRNA and DNA vaccines create new variants that selectively avoid the partial immunity provided by such vaccines.

EduCaps™ avoid the possibility of new pandemic viral emergencies, cytokine storms, mitochondria aerobic failure, serious illnesses, or death via variant strains of COVID-19. Thus, EduCaps™ provide a safe dendritic human non-antigenic encapsulated vaccine that transfers recognition of the immunogenic substance/virus to the human immune system.

The present invention blocks and terminates COVID-19 viruses (or variants) from attaching to the ACE2 receptor of the human Sigma One RBD antigen, thereby blocking viral entry into human cells by COVID-19 or any variant. Since the Sigma One RBD antigen is common to ALL COVID variants that can affect humans, EduCaps™ do not cause variants that can spread or become selectively dominant as a result of EduCap™ vaccination.

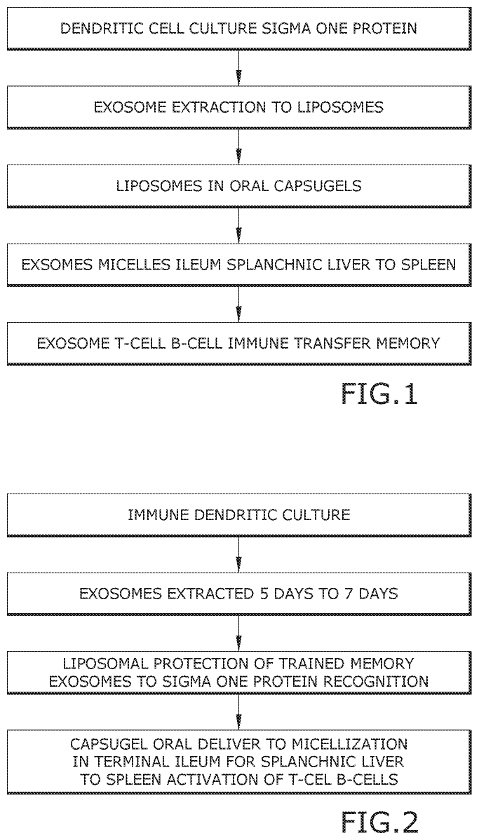

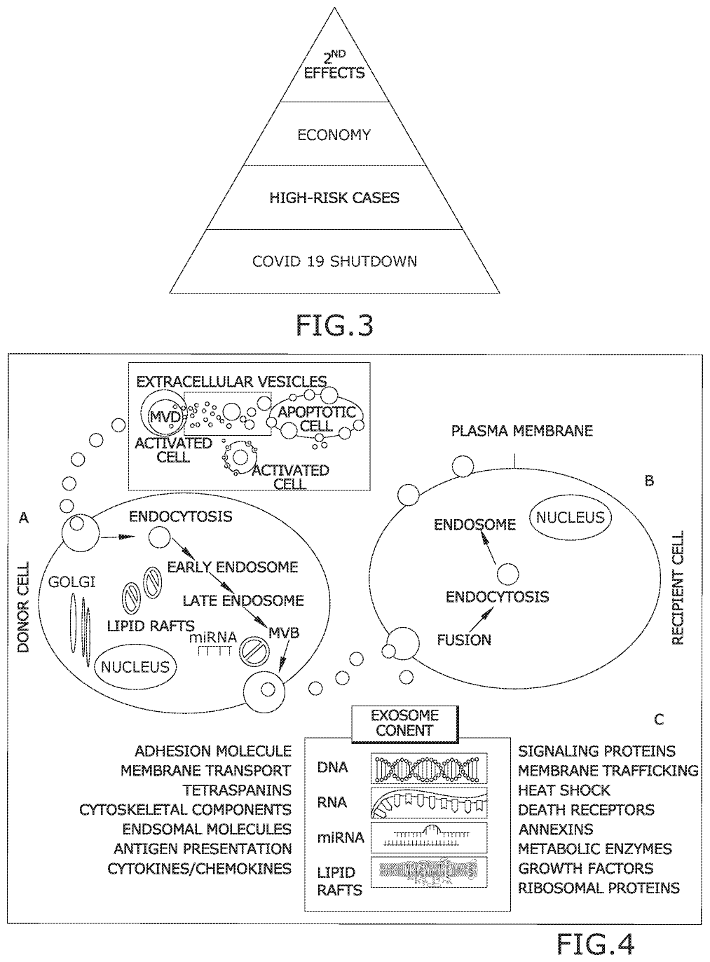

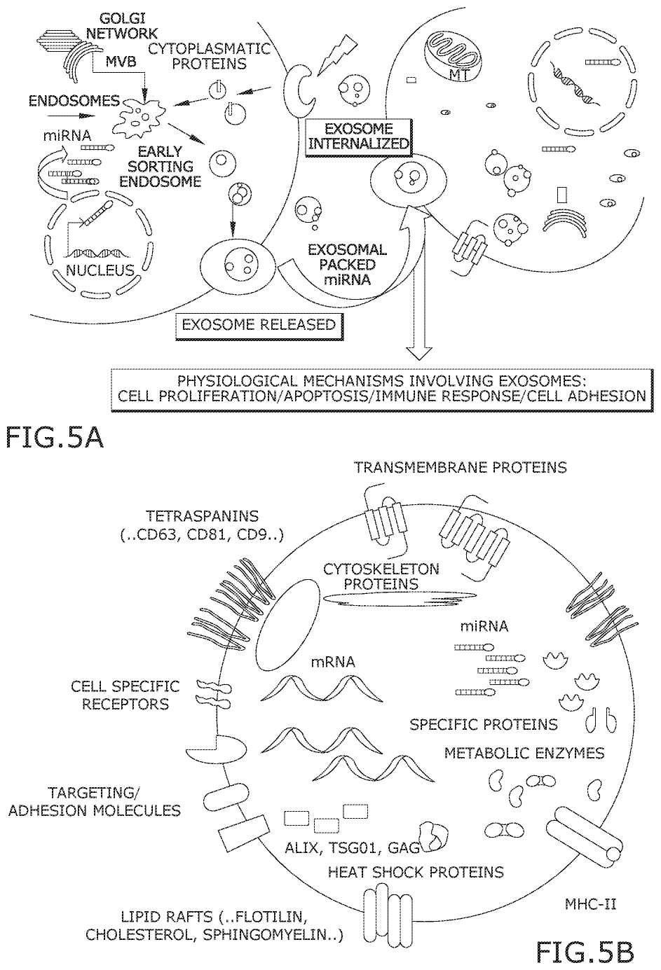

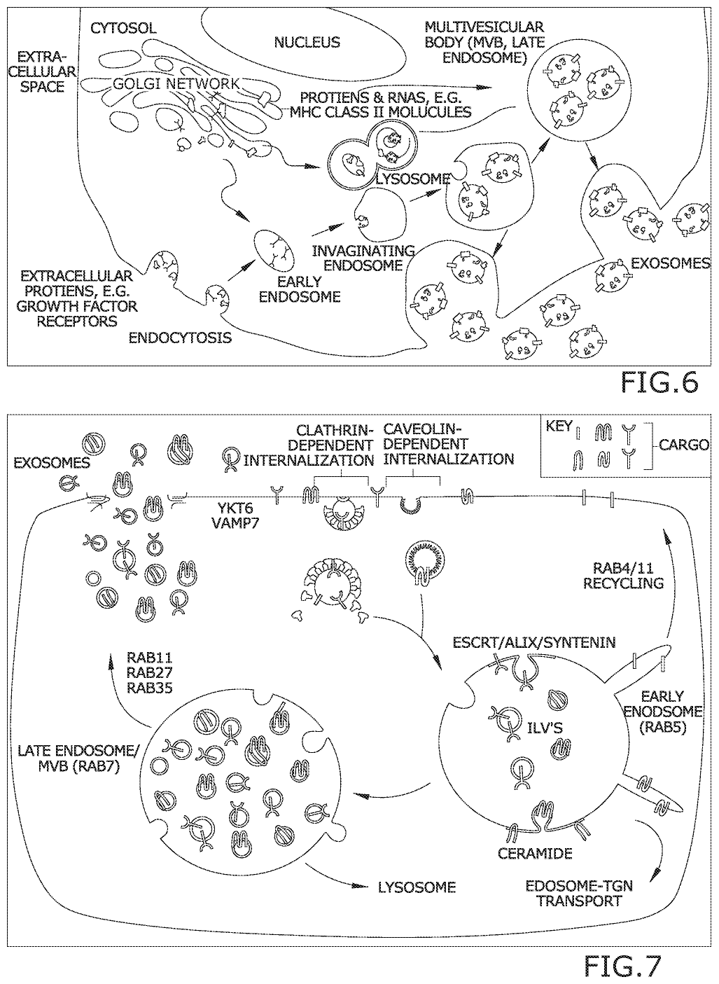

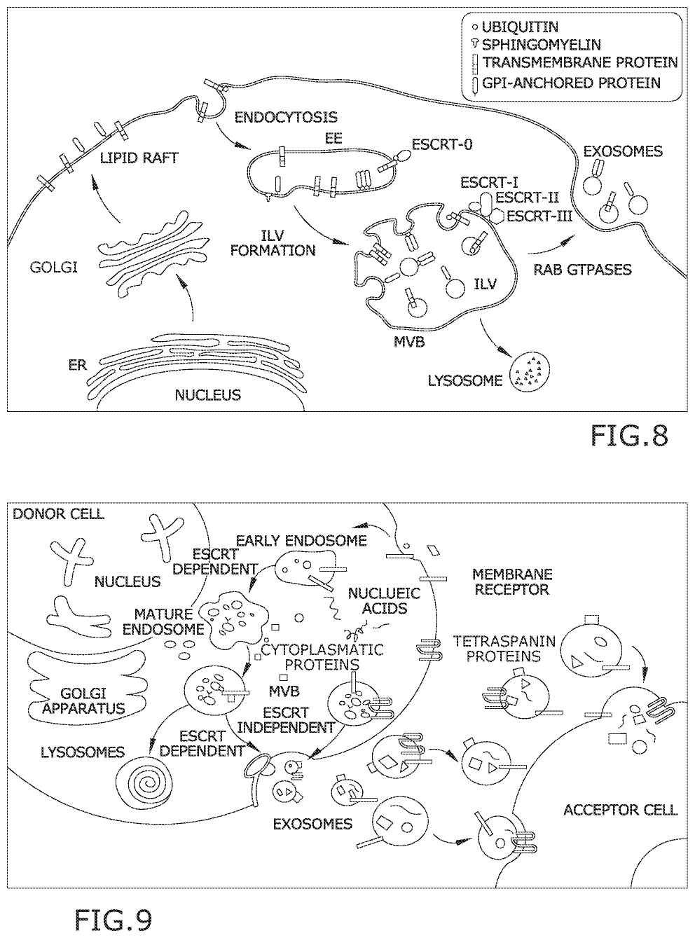

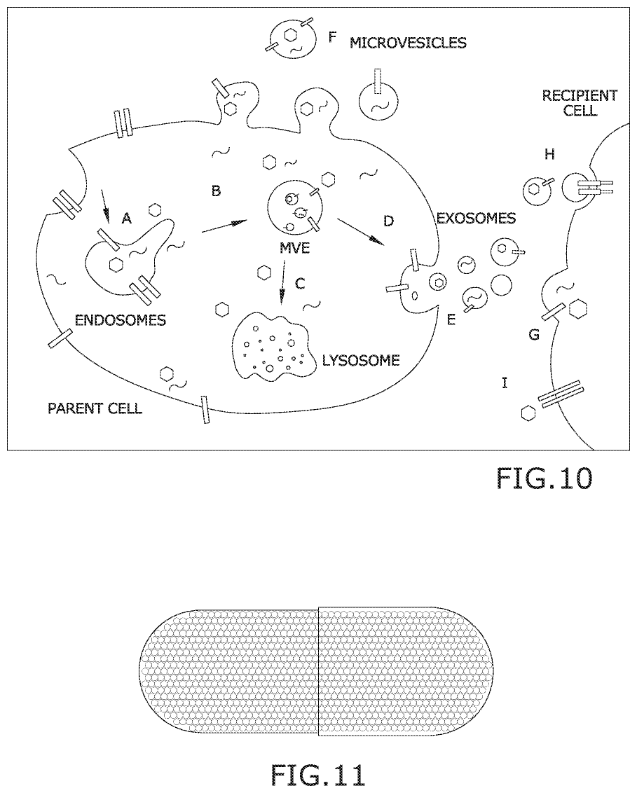

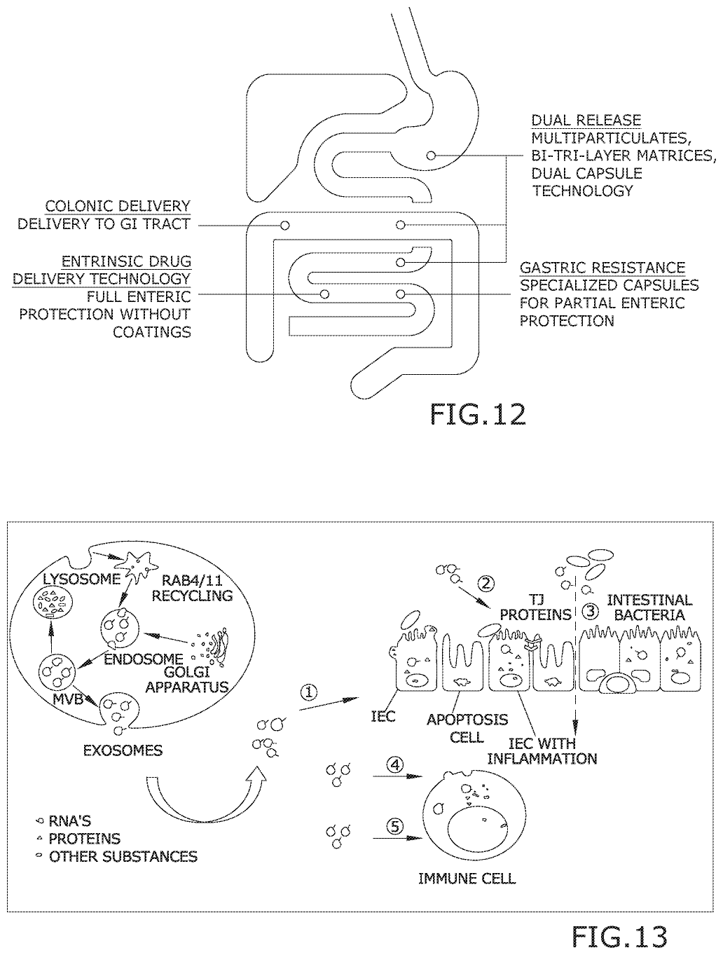

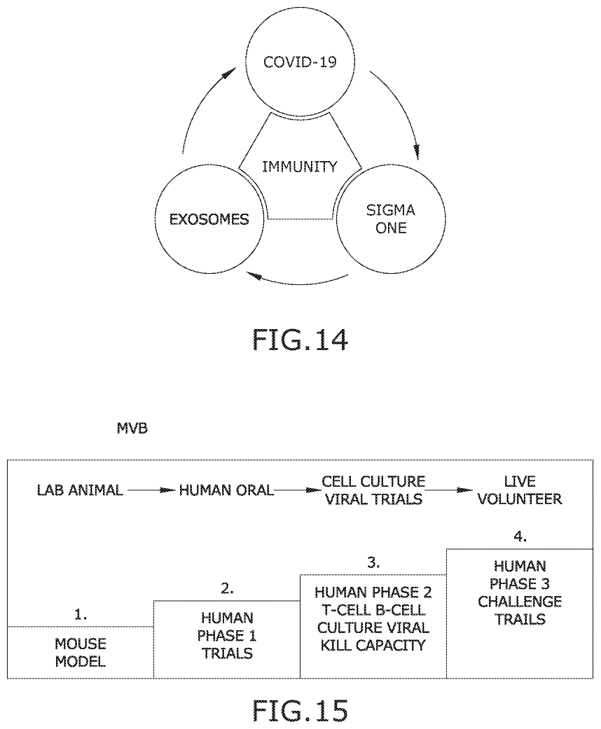

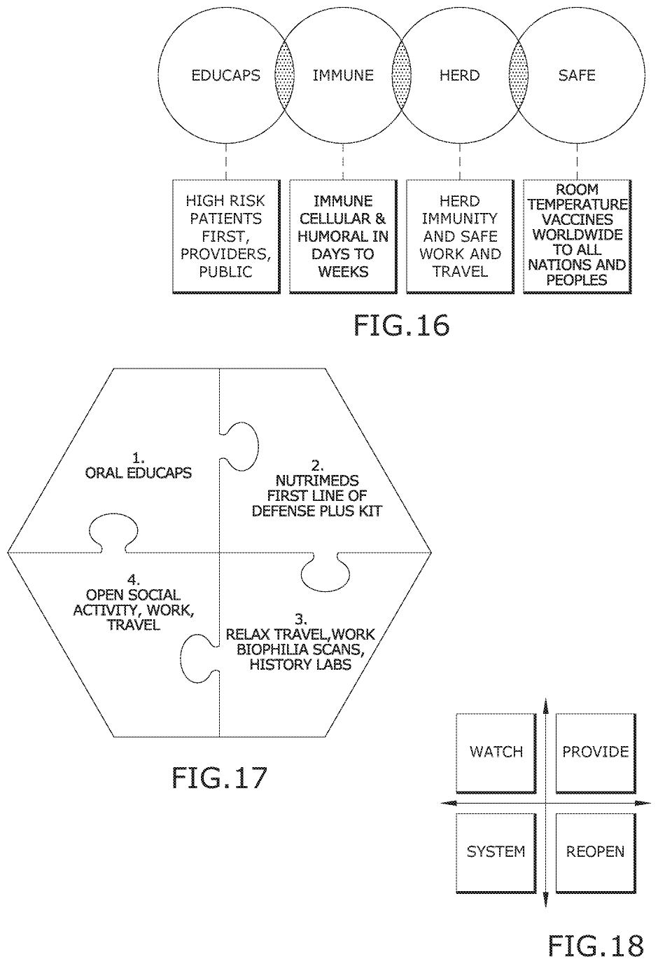

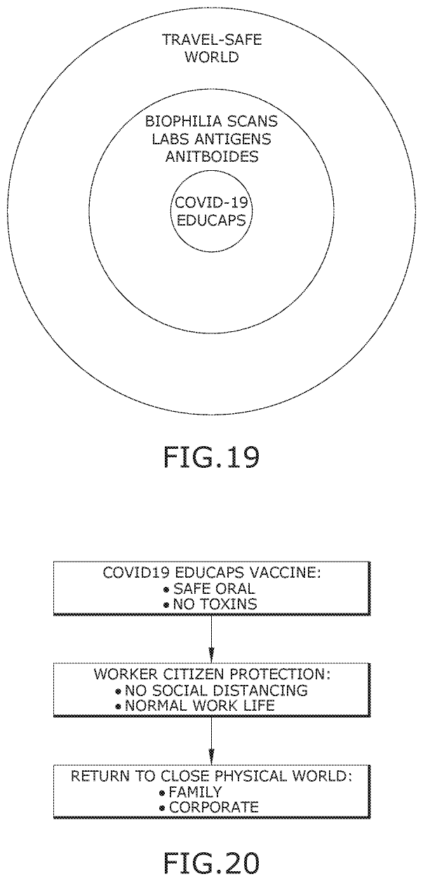

Referring now to FIGS. 1 through 20, the present invention includes a method and system of programming immune memory molecules for whole body protection. The process incorporates the following steps: step 1, immune dendritic culture of Sigma One protein of COVID-19; step 2, exosome miRNA memory molecule extraction (five to seven days); step 3, protection of trained memory (solution of) exosomes Sigma One protein recognition—e.g., including freeze-drying the exosomes, and then enclosing them in Delayed Release (DR) oralcapsules; step 4, taking the Delayed Release oral capsules containing the freeze-dried exosome-containing miRNA EduCaps™ via oral ingestion (e.g., swallowing) (EduCaps™ are stable at room temperature, taken with water, juice, or fluids) the exosomes containing miRNA to be micellized at the terminal ileum and then absorbed for entry into the splanchnic circulation to the liver sinusoids, the spleen, and the bone marrow to train the T-cells and B-cells residing therein; and step 5, monitor blood immune T-cell cellular and B-cell immunoglobulin response in 4 to 6 weeks (including blood labs immunological assay) for verification of immune transfer memory effectuation. This educates the immune system, particularly, IgG4 T-cell virus killing and B-cell viral tagging IgM antibodies. By performing lab testing at two to four weeks after consuming the oral EduCaps™, detectable antibodies can be measured that indicate successful immune memory transfer. Immune memory transfer includes transfer of miRNA (microRNA) that contain information that enables IgG4 T-cell viral killing capacity, and B-cell viral identification capacity. This creates long term T-cell and B-cell immunity, thereby providing complete viral illness protection.

Antigen transfer to dendritic cells creates exosomes that educate the immune system to recognize COVID-19 and variants, and to identify and kill the COVID-19 virus attaching to the ACE2 receptor.

Thus, immune memory molecules (microRNA) are contained within exosomes, which are dried and then encapsulated for pH protection from stomach acid and digestive enzymes (e.g., using Lonza® Capsugel® DR™ capsules). Dendritic exosomes transfer immune information to T-cells and Bells throughout the body, thereby providing to the person consuming the EduCaps™ cellular and humoral immune protection from COVID-19, and all variants thereof.

The present invention can be manufactured using a bioreactor with standardized universal human dendritic cells, and standardized Spike Protein antigen for incubation therewith. The standardized universal human dendritic cells and Spike Protein together after 5-7 days of incubation, generate immune-training exosomes effective against COVID-19 and variants. The immune-training exosomes are then freeze-dried, and then encapsulated within Delayed Release Capsules (such as LONZA® Capsugel® DR™ capsules) without any binders or adjuvants, thereby providing oral EduCaps™. The oral EduCaps™ of the invention provide symptom-free induction of immunity to COVID-19 and all variants thereof, without infection by COVID-19 or variants, and without symptoms of COVID-19 or variants.

A user can orally consume one EduCap™ capsule for each of seven days, with water or juice, thereby providing the user with immune education against COVID-19 or any variant thereof, without suffering any toxicity or side effects, and without experiencing any symptoms of COVID-19 or any variant thereof.

It should be understood, of course, that the foregoing relates to exemplary embodiments of the invention, and that modifications can be made without departing from the spirit and scope of the invention as set forth in the following claims.

1. A method of immunizing a subject against a molecule-caused disease without introducing an antigen associated with the molecule-caused disease, the method comprising:
administering an effective amount of one or more exosomes to the subject, wherein each exosome is isolated from a dendritic culture of sigma one protein incubated with an immunogenic substance associated with the molecule-caused disease.

2. The method of claim 1, wherein said effective amount is encapsulated.

3. The method of claim 1, further comprising orally administering an encapsulated effective amount to target intestinal antigen-presenting cells.

4. A composition for immunizing against a molecule-caused disease, the composition comprising:
one or more exosomes isolated from a dendritic culture of sigma one protein incubated with an immunogenic substance associated with the molecule-caused disease.
